(19) United States
(12) Patent Application Publication
(10) Pub. No.: US 2004/0081601 A1
(43) Pub. Date: Apr. 29, 2004

(54) Integrity testable autoclavable, sterile transfer bag

(76) Inventors: Martin Morrissey, Beverly, MA (US); Stephen Proulx, Boxboro, MA (US)

Correspondence Address: MILLIPORE CORPORATION, 290 CONCORD ROAD, BILLERICA, MA, 01821, US

(21) Appl. No.: 10/659,570

(22) Filed: Sep. 9, 2003

Related U.S. Application Data
(60) Provisional application No. 60/421,909, filed on Oct. 29, 2002.

(51) Int. Cl. A61L 2/07; B65D 1/09

(52) U.S. Cl. 422/294; 422/26; 206/528

(57) ABSTRACT

The present invention is formed of a transfer bag that is capable of being sterilized by steam or gas and then sealed to form an integral sterile device. It is preferably attached to a closed collar designed to fit onto a transfer port. The bag is formed of two sections, a first steam penetrable, porous section and a second non-porous section. The items are loaded into the bag through an opening; it is sealed and exposed to a steam or sterilizing gas environment. Steam or gas enters the first section of the bag and moves throughout the interior of the bag sterilizing the bag and its contents. After sterilization, the bag is removed and the contents are moved so as to all be within the second section of the bag. The bag is then sealed adjacent to the interface between the first and second sections to ensure the sterility of the bag and its contents. At the same time or shortly thereafter, a vacuum or positive pressure is applied to the second portion of the bag to serve as a visual indication that the sterilizing process has occurred and that the sterility of the bag and its contents has been maintained.

The present invention relates to a transfer bag for a sterile chamber transfer port. More particularly, it relates to a transfer bag for a transfer port, which is autoclavable and integrity testable.

### BACKGROUND

The use of sterile rooms, such as for the filling of pharmaceuticals and biopharmaceuticals has gained and is continuing to gain acceptance.

These rooms are isolated from the outside atmosphere and environment by various plastic, glass and metal barriers. They often are in the form of a glove box.

Transfer ports are used to provide a means for allowing items in to and out of the isolator.

U.S. Pat. No. 6,030,578 shows one preferred transfer port, sold as the Safe Pass™ system by Millipore Corporation of Bedford, Mass.

The system comprises a movable door mounted by its mating port to an opening of an isolator. A transfer bag containing the items to be passed into the isolator is attached by its collar to the port. The items inside have been pre-sterilized such as by gamma radiation. After the bag is attached, sterilizing light in the form of UV or pulsed white light is shown upon the exposed surfaces of the mated collar to eliminate any potential microorganisms that would otherwise contaminate the chamber upon opening of the port.

After sterilization, the port is opened exposing the sterilized face of the collar to the interior of the isolator.

The collar face is removed or pierced to allow one to have access to the items in the transfer bag for use in the isolator.

After use, the items are sealed within the isolator (for example: a pharmaceutical is filled into vials or syringes in the isolator, sealed to keep their sterility and then place into the transfer bag attached to the open port). The port door is then closed to maintain the sterility of the isolator and the bay is disconnected and taken away for further processing of sale.

One current limitation is that the bag and its contents must be sterilized by gamma radiation.

A second limitation with the current system is that the bag and its contents, either before of after sterilization, are subjected to a slight vacuum or positive pressure, which is then sealed and maintained.

This provides one with a reliable and simple integrity test. So long as the package maintains the slight vacuum or pressure, the user knows the contents inside are sterile. If the vacuum or pressure is lost, integrity of the bag has been compromised and is visibly indicated as such and the bag and its contents are not allowed into the isolator.

While the system works, it is desired to eliminate the use of radiation as the means for sterilizing the bag and its contents.

What is desired is a system that allows one to use autoclaving to sterilize the contents of the bag. It is also desired to be able to do so while still being able to utilize the vacuum or pressure integrity mechanism for the integrity test.

The present invention provides such a device.

### SUMMARY OF THE INVENTION

The present invention is formed of a transfer bag that can be sterilized by a gas or steam, sealed so as to eliminate the further entrance or gas or vapor and then subjected to a slight vacuum or positive pressure to provide a visual indication of the integrity of the seal. It may be attached to a closed collar designed to fit onto a transfer port. The bag is formed of two zones, a first steam penetrable, porous section and a second non-porous sterile section. The items are loaded into the bag through its opening; it is sealed and exposed to a sterilizing gas or an autoclave environment. Steam or gas such as ethylene oxide enters the first portion of the bag and moves throughout the interior of the bag sterilizing the bag and its contents. After sterilization, the bag is removed and the contents are moved so as to all be within the second portion of the bag. The bag is then sealed adjacent to the interface between the first and second portions to ensure the sterility of the bag and its contents. At the same time or shortly thereafter, a vacuum or positive pressure is applied to the second portion of the bag to indicate that the sterilizing process has occurred and to act as a visual indicator of the sterility of the bag and its contents.

It is an object of the invention to provide a transfer bag comprising a bag having a first porous section and a second non-porous section, wherein after sterilization by a means selected from the group consisting of steam and sterilizing gas, the contents are moved to the second non-porous section of the bag and then the bag is sealed adjacent to an interface between the first and second portions to form a sterile, sealed region.

It is another object of the invention to provide a transfer bag comprising a bag having a first porous section and a second non-porous section, a closed collar designed to fit onto a transfer port attached to the non-porous section of the bag wherein the bag contains components to be sterilized and transferred to an isolator.

It is an additional object of the invention to provide a transfer bag comprising a bag having a first porous section and a second non-porous section, a closed collar designed to fit onto a transfer port attached to the non-porous section of the bag wherein the bag contains sterile components having been steam sterilized in the bag and the bag contains a seal in the non-porous section adjacent to an interface between the first and second sections to ensure the sterility of the bag and its contents.

It is a further object of the invention to provide a transfer bag comprising a bag having a first porous section and a second non-porous section, a closed collar designed to fit onto a transfer port attached to the non-porous section of the bag wherein the bag contains sterile components having been steam sterilized in the bag and the bag contains a seal in the non-porous section adjacent to an interface between the first and second sections to ensure the sterility of the bag and its contents and the bag and its contents being under a slight vacuum or positive pressure to provide a visual indicator of the integrity of the bag and seal.

It is another object of the invention to provide a transfer bag comprising a bag having a first porous section and a second non-porous section, a closed collar designed to fit onto a transfer port attached to the non-porous section of the bag wherein the first section of the bag forms a portion of a first surface of the bag.

It is an additional object of the invention to provide a transfer bag comprising a bag having a first porous section and a second non-porous section, a closed collar designed to fit onto a transfer port attached to the non-porous section of the bag wherein the bag has two surfaces and the first section of the bag forms a portion of both surfaces of the bag.

It is a further object of the invention to provide a transfer bag comprising a bag having a first porous section and a second non-porous section, a closed collar designed to fit onto a transfer port attached to the non-porous section of the bag wherein the bag has two surfaces, the first section of the bag forms a portion of both surfaces of the bag and the first section of each surface is in register with each other.

It is an object of the invention to provide a transfer bag comprising a bag having a first porous section and a second non-porous section, a closed collar designed to fit onto a transfer port attached to the non-porous section of the bag wherein the bag has two surfaces and a first and second end, the first section of the bag forms a portion of both surfaces of the bag at the first end of the bag and the first section of each surface is in register with each other.

It is another object of the invention to provide a transfer bag comprising a bag having a first porous section and a second non-porous section, a closed collar designed to fit onto a transfer port attached to the non-porous section of the bag wherein the bag has two surfaces and a first and second end, the first section of the bag forms a portion of one surface of the bag at the first end of the bag.

It is an additional object of the invention to provide a transfer bag comprising a bag having a first porous section and a second non-porous section, a closed collar designed to fit onto a transfer port attached to the non-porous section of the bag wherein the bag contains sterile components having been steam sterilized in the bag and the bag contains a seal in the non-porous section adjacent to an interface between the first and second sections to ensure the sterility of the bag and its contents and the bag containing a vacuum port in its second section so that the bag and its contents are under a slight vacuum.

It is an additional object of the invention to provide a transfer bag comprising a bag having a first porous section and a second non-porous section, a closed collar designed to fit onto a transfer port attached to the non-porous section of the bag wherein the bag contains sterile components having been steam sterilized in the bag and the bag contains a seal in the non-porous section adjacent to an interface between the first and second sections to ensure the sterility of the bag and its contents and the bag containing a pressure port in its second section so that the bag and its contents are under a slight positive pressure.

It is a further object of the invention to provide a process for sterilizing and creating a visual indication of integrity for a bag comprising the steps of providing a bag having two sections, a first porous section and a second non-porous section, an open end and a closed end, filling the bag with a selected component, forming a first seal in the bag at the open end, subjecting the bag and its contents to sterilizing gas or vapor such as ethylene oxide gas or steam for a period of time sufficient to sterilize the contents, moving the contents to the second section of the bag and forming a second seal in the non-porous section between the contents and the porous section.

### IN THE DRAWINGS

FIG. 1 shows a first embodiment of a bag according to the present invention in cross section view.

FIG. 2 shows the embodiment in FIG. 1 after sterilization and the formation of the seal and slight vacuum to indicate integrity.

FIG. 2A shows the embodiment in FIG. 1 after sterilization and the formation of the seal and slight positive pressure to indicate integrity.

FIG. 3 shows the embodiment of FIG. 2 in use on a transfer port in a cross sectional view.

FIG. 4 shows a second embodiment of a bag according to the present invention in cross section view.

FIG. 5 shows a method of using a bag according to the present invention in block diagrams.

FIG. 6 shows a third embodiment of a bag according to the present invention in cross section view.

### DETAILED DESCRIPTION OF THE INVENTION

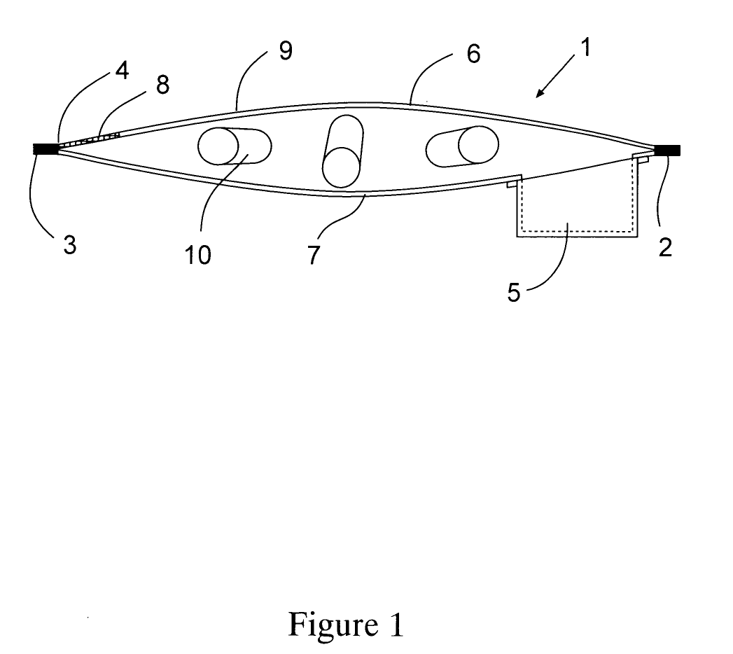

FIG. 1 shows a first embodiment of the present invention. The bag 1 has a first closed end 2 and a second end 3 that is supplied in an open format and sealed by the user after insertion of the desired contents. FIG. 1 shows the bag in a sealed condition with seal 4 at the second end 3 of the bag 1. The bag in this embodiment also contains a collar 5 which is closed and is used to attach the bag to a transfer port. The collar may be one such as is used on a sterile transfer port known as SAFEPASS™ system available from Millipore Corporation of Bedford, Mass. (See U.S. Pat. No. 6,030,578 and U.S. Pat. No. 6192,948) or it may form one of the flanges of the more conventional Alpha/Beta ports which typically rely on aspectic swabbing of the collar or flange containing the bag to ensure a sterile transfer although some Alpha/Beta ports have been mentioned as being capable of having light or dry heat driven sterility capabilities. The bag 1 has two major surfaces or sides 6, and 7. At least one surface, in this instance surface 6 is formed of two portions, a first portion 8 and a second portion 9.

The first portion 8 is formed of a porous, sterilizing grade filter material such as a non-woven material (Tyvek® porous sheet material available from Dupont) or a microporous filter material such as a hydrophilic microporous PVDF membrane (Durapore® membrane available from Millipore Corporation of Bedford, Mass.). Such materials are well known in the industry and typically have a nominal pore of less than about 0.22 microns (the size of the smallest bacteria). It may be of a nominal pore size of from about 0.05 to about 0.22 microns in one embodiment. It is often of a nominal pore size of from about 0.1 micron to about 0.22 micron.

This porous material is accepted in the industry as being able to gas such as ethylene oxide and steam, but is able to form a sterile barrier against the movement of bacteria, molds and yeasts into the container.

The first portion 8 is bonded to a second portion 9 of the bag 1, which is made of a non-porous material. In one embodiment, the non-porous material of the second portion 9 is a plastic. Suitable plastics include but are not limited to polyolefins such as polyethylene and polypropylene, PET, polyester films, such as Mylar® film, PVDF, PES, polysulfone, polyethersulfone, polyarylsulfone, polyphenylsulfone, PVC, acrylic resins, methacrylic resins, EVA copolymers, EVOH and blends, metallized versions of these materials, laminates and composites of any of the above.

The second portion 9 may be a single layer or formed as a laminate or co-extrusion of two or more layers, which may be of the same or different materials.

The first portion 8 should account for at least a portion of one surface 6 of the bag, the amount used being set by a number of parameters including the length of the gas or steam sterilization cycle, the pressure of the gas or steam, the rate of transfer of the sterilizing gas or steam through the first portion, the size of the bag and the like. Generally it should be sufficient to allow gas or steam to enter the bag and fully sterilize all of its contents within the normal cycle parameters for the contents of the bag. In one embodiment, it is from about 5% to about 100% of one surface of the bag. In another, it is from about 10% to about 50% of one surface of the bag. In a further embodiment, it is from about 10% to about 25% of one surface of the bag.

As shown in FIG. 1, the second surface 7 is formed of a non-porous film, preferably of the same material as the second portion 9 of the first side 6. The first and second sides, 6,7 are sealed along their adjoining edges.

A bag according to FIG. 1 is supplied with the end 3 in an unsealed condition. A user loads the bag with the product 10 to be sterilized and then forms the seal 4 by heat, vibration welding such as ultrasonic welding, by adhesives or solvents that cause the two layers to seal together at the end 3 or by a mechanical means such as a bar clamp or the like.

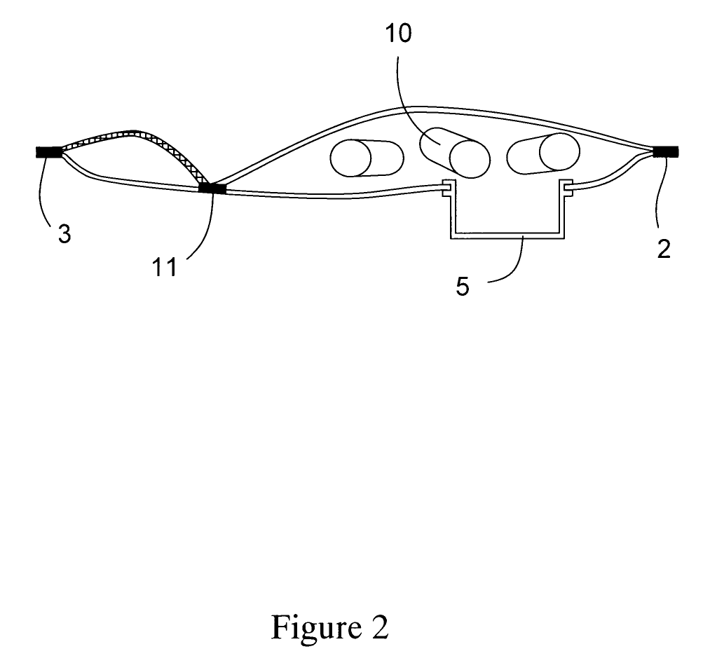

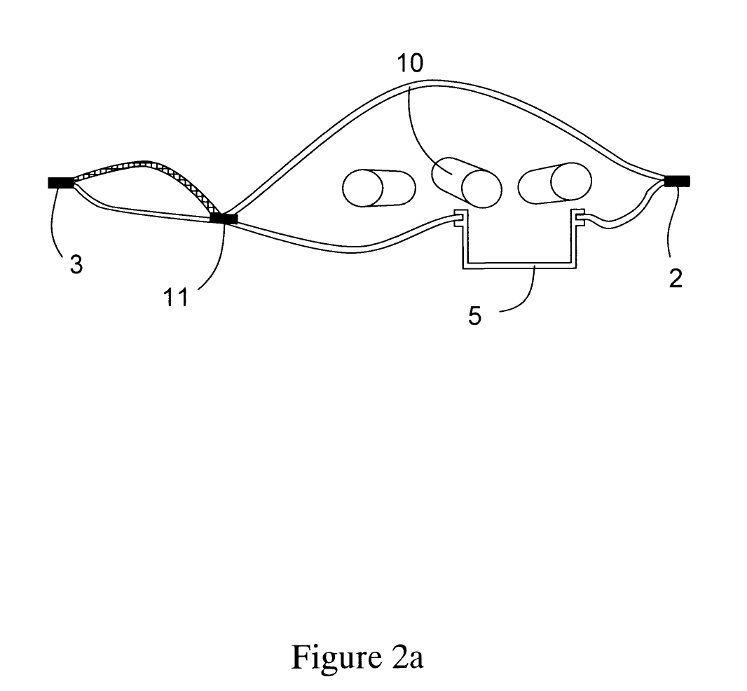

The bag and its contents are then placed in a chamber, preferably an autoclave in the case of steam, and exposed to sterilizing gas, vapor or steam, preferably at an elevated pressure for a time sufficient to sterilize the contents. The bag in then removed and the contents are all moved to the portion of the bag formed by the non-porous materials such as by tipping the bag or physically moving the contents from the outside of the bag by hand and the like. A seal 11 is then formed in the non-porous section of the bag adjacent to the porous area so that the contents are now sealed within a non-porous bag as shown in FIG. 2. The bag may be subjected to a slight vacuum or positive pressure during the seal formation applied through the porous material or by compression of the non-porous section to reduce the amount of air with in that section. Alternatively, one may provide a vacuum/pressure port to allow the vacuum/pressure to be formed in the sealed bag 1 either during or after sealing.

FIG. 2 shows the embodiment where a slight vacuum is applied to cause the bag to slightly shrink. FIG. 2A shows a similar bag in which a slight positive pressure has been used to inflate the bag. Both give one a visual indication of the integrity of the bag. If the seal is lost on the vacuum bag it is clearly visible by the non-compressed condition of the bag. Likewise, if the positive pressure is lost, the bag appears to be deflated. Both indicate to the operator a compromise of eth integrity of the bag such as a puncture of the bag during its storage or handling or the loss of the seal between the porous portion and the non-porous portion during assembly, handling or storage.

The use of a dedicated port in the bag by which a slight vacuum or positive pressure is applied also has advantages in integrity testing before use in that one can use the port to perform various integrity tests on the bag such as pressure holding, pressure decay, vacuum holding or decay studies to ensure that the bag is integral and that no leaks have been formed in the seal or the bag itself.

Before use, the bag is either visually inspected to ensure that the vacuum or pressure is still being applied or tested with a integrity test such as a pressure or vacuum holding test described above. If it is found to be integral, one can use the bag as its contents are then still sterile. If the vacuum or pressure has been compromised, one should not use the bag, remove the contents and place them in a new bag and sterile the contents before using.

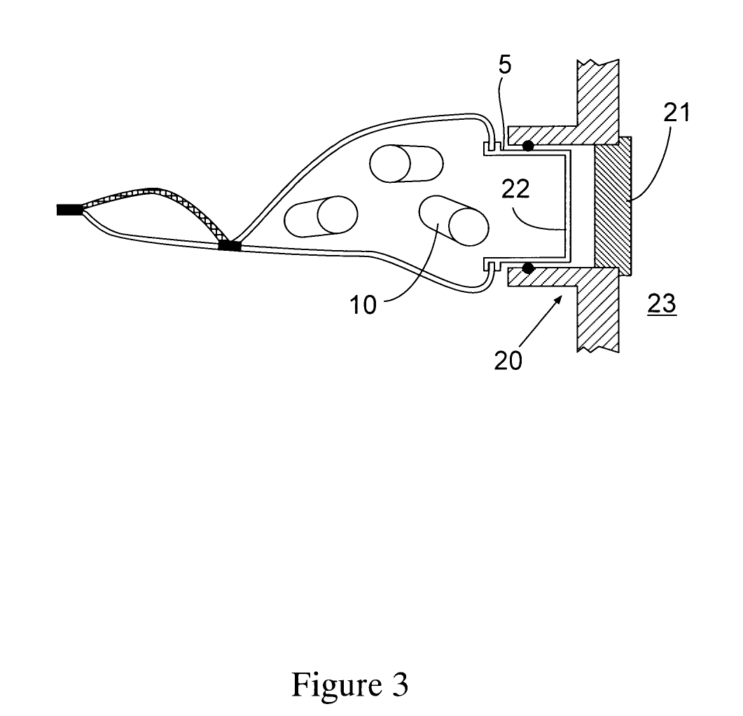

The bag 1 is attached by the collar 5 to the transfer port 20 as shown in FIG. 3. The surface of the collar 5 may be sterilized with light such as UV or pulsed light, dry heat, steam, microwave energy, aseptic swabs of alcohol and the like before the door 21 is opened. The collar face 22 is then either cut open or removed allowing one inside the chamber 23 to pull the sterile contents of the bag 1 into the chamber 23 for use.

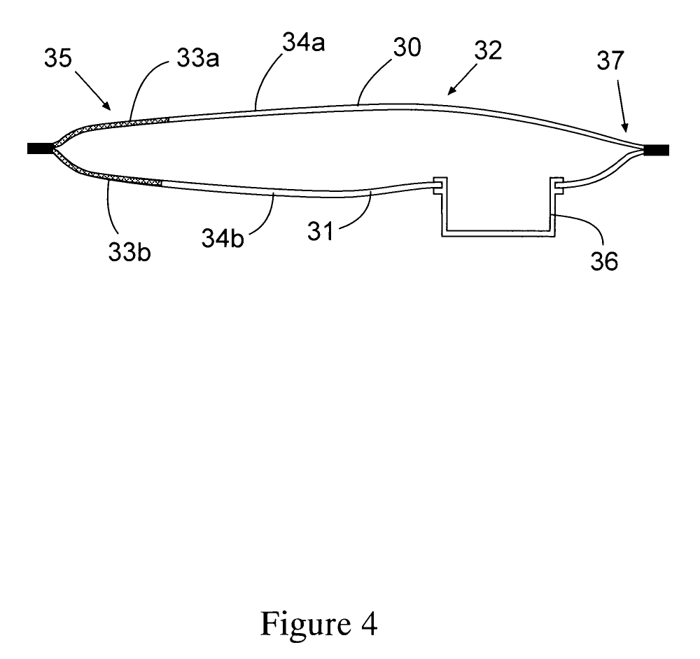

FIG. 4 shows a second embodiment of the present invention. In this embodiment, both surfaces 30 and 31 of a bag 32 contain a porous section 33a and b and a non-porous sections 34a and b. Porous sections 34a and b are arranged across from each other (or in register with each other) as are porous sections 33a and b. Preferably, as shown the porous section 33a and b are formed at one end 35 of the bag 32. A collar 36 is mounted to one surface 30 of the bag 32. As shown, it 36 is attached near the other end 37 of the bag 32. Alternatively, it may be attached to any non-porous section of the bag. The materials for the sections are the same as those of FIG. 1.

The amount of porous material is can be the same on each surface or different amounts can be used on each side. The amount used on each side and in total is set by a number of parameters including the length of the sterilization cycle, the pressure of the gas or steam, the rate of transfer of gas or steam through the first portion, the size of the bag and the like. Generally, it should be sufficient to allow gas or steam to enter the bag and fully sterilize all of its contents within the normal gas or steam sterilization cycle for the contents of the bag. In one embodiment, it is from about 5% to about 70% of the surface area of the bag. In another, it is from about 5% to about 50% of the surface area of the bag. In a further embodiment, it is from about 10% to about 50% of the surface area of the bag. In another embodiment, it is from about 10% to about 25% of the surface area of the bag.

The bag as supplied to the customer has one end or side that is open so that contents may be placed inside the bag. The open portion is then sealed as described above and the bag is then sterilized with steam or gas that passes through the porous sections. After sterilization, the contents of the bag are all moved to the non-porous section such as by tipping the bag or pushing the contents into that section of the bag by hand from the outside of the bag and the bag is sealed at a location between the porous and non-porous sections, preferably in the non-porous section adjacent the interface between the porous and non-porous sections.

As with the embodiment of FIG. 1, a vacuum or positive pressure can be applied during the sealing through the porous section or via a separate vacuum/pressure port or by compression of the non-porous sections to either vent gas out through the porous section before sealing or to trap positive pressure within the non-porous area before the seal is made. Regardless of when and how it is done, it forms a visible integrity check of the bag's condition. The bag is then mounted and used in the same manner as that described above in relation to FIG. 3.

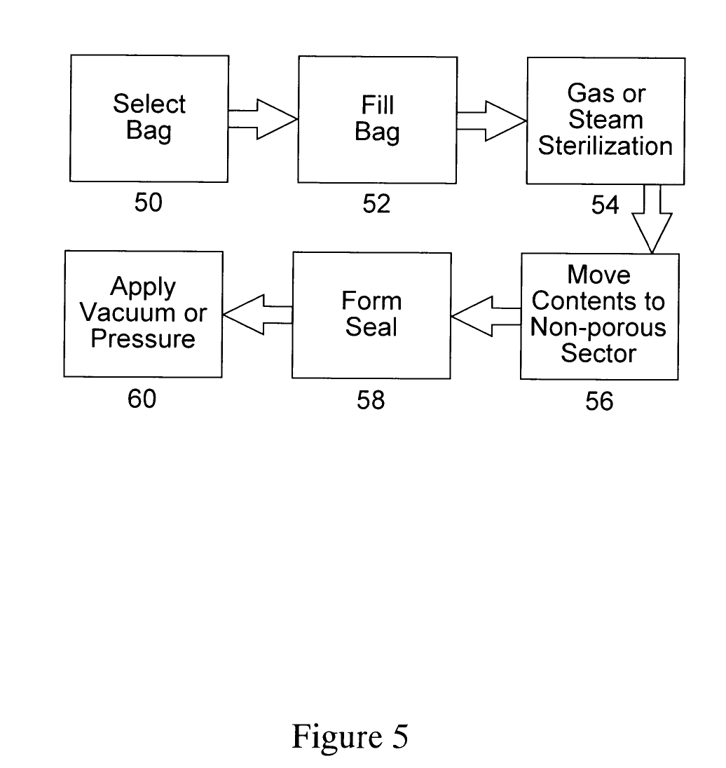

FIG. 5 shows a preferred method for sterilizing and forming a bag with a visible integrity check. The process consists of a first step 50 of taking a bag having a first porous section and a second non-porous section according to the invention and filling it 52 to the desired level with products to be sterilized. Sterilizing the bag and its contents by gas or steam is the next step 54. The bag is then tipped 56 and a seal is formed 58 between the first section and second section. A vacuum or positive pressure is applied 60 to the non-porous section either during or after the sealing between the sections to complete the sterile, integrity indicator bag.

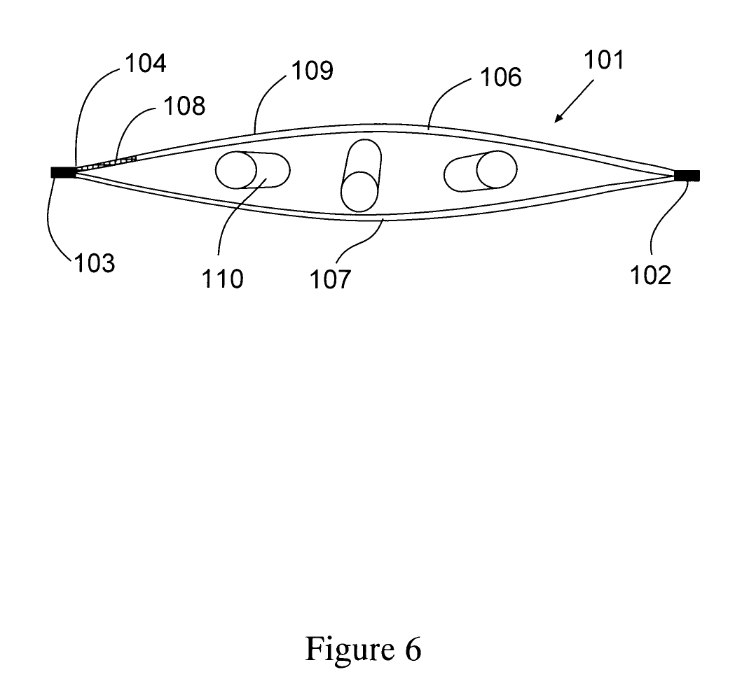

The bag also has applications in other fields such as medicine. In such an embodiment, the collar of the previous figures in typically not needed as is shown in FIG. 6. Here the bag is similar to that of FIG. 1 with the exception no collar is formed as part of the bag. The bag 101 has a first closed end 102 and a second end 103 that is supplied in an open format and sealed by the user after insertion of the desired contents. FIG. 6 shows the bag in a sealed condition with seal 104 at the second end 103 of the bag 101. The bag 101 has two major surfaces or sides 106 and 107. At least one surface, in this instance surface 106 is formed of two portions, a first portion 108 and a second portion 109. As shown the first portion 108 is formed of a microporous material that allows for the use of steam or gas to sterilize the contents of the bag 101 before the seal 104 is formed.

What is claimed:

1) A transfer bag comprising a bag having a first porous section and a second non-porous section, wherein after sterilization by a means selected from the group consisting of steam and sterilizing gas, the contents are moved to the second non-porous section of the bag and then the bag is sealed adjacent to an interface between the first and second portions to form a sterile, sealed region.

2) The transfer bag of claim 1 wherein the bag further comprises a closed collar attached to the non-porous section of the bag that is designed to fit onto a transfer port.

3) The transfer bag of claim 1 wherein the bag contains components to be sterilized and transferred to an isolator.

4) The transfer bag of claim 1 wherein the bag contains sterile components having been steam sterilized in the bag and the bag contains a seal in the non-porous section adjacent to an interface between the first and second sections to ensure the sterility of the bag and its contents.

5) The transfer bag of claim 1 wherein the bag contains sterile components having been steam sterilized in the bag and the bag contains a seal in the non-porous section adjacent to an interface between the first and second sections to ensure the sterility of the bag and its contents and the bag and its contents being under a slight vacuum.

6) The transfer bag of claim 1 wherein the bag contains sterile components having been steam sterilized in the bag and the bag contains a seal in the non-porous section adjacent to an interface between the first and second sections to ensure the sterility of the bag and its contents and the bag and its contents being under a slight positive pressure.

7) The transfer bag of claim 1 wherein the bag contains sterile components having been steam sterilized in the bag and the bag contains a seal in the non-porous section adjacent to an interface between the first and second sections to ensure the sterility of the bag and its contents and the bag containing a pressure port in its second section so that the bag and its contents are under a slight positive pressure.

8) The transfer bag of claim 1 wherein the first section of the bag forms a portion of a first surface of the bag.

9) The transfer bag of claim 1 wherein the bag has two surfaces and the first section of the bag forms a portion of both surfaces of the bag.

10) The transfer bag of claim 1 wherein the bag has two surfaces, the first section of the bag forms a portion of both surfaces of the bag and the first section of each surface is in register with each other.

11) The transfer bag of claim 1 wherein the bag has two surfaces and a first and second end, the first section of the bag forms a portion of both surfaces of the bag at the first end of the bag and the first section of each surface is in register with each other.

12) The transfer bag of claim 1 wherein the bag has two surfaces and a first and second end, the first section of the bag forms a portion of one surface of the bag at the first end of the bag.

13) The transfer bag of claim 1 wherein the bag contains sterile components having been steam sterilized in the bag and the bag contains a seal in the non-porous section adjacent to an interface between the first and second sections to ensure the sterility of the bag and its contents and the bag containing a vacuum port in its second section so that the bag and its contents are under a slight vacuum.

14) The transfer bag of claim 1 wherein the first section of the bag is formed of a material selected from the group consisting of non-wovens and microporous materials.

15) The transfer bag of claim 1 wherein the second section of the bag is formed of a plastic selected from the group consisting of polyolefins, PET, polyester, PVDF, PES, polysulfone, polyethersulfone, polyarylsulfone, polyphenlysulfone, PVC, acrylic resins, methacrylic resins, EVA copolymers, EVOH and blends, metallized versions of the above, laminates and composites of any of the above.

16) A process for sterilizing and creating an indication of integrity comprising the steps of providing a bag having two sections, a first porous section and a second non-porous section, an open end and a closed end, filling the bag with a selected component, forming a first seal the bag at the open end, subjecting the bag and its contents to steam for a period of time sufficient to sterilize the contents, moving the contents to the second section of the bag and forming a second seal in the non-porous section between the contents and the porous section.

17) The process of claim 16 further comprising the step of applying a vacuum to the second section during or after the formation of the second seal.

18) The process of claim 16 further comprising the step of applying a positive pressure to the second section during or after the formation of the second seal.

19) The process of claim 16 further comprising the step of applying an integrity test the bag after formation of the second seal.

20) The process of claim 16 further comprising the step of applying an integrity test the bag after formation of the second seal wherein the test is selected from the group consisting of pressure holding, pressure decay, vacuum holding and vacuum decay test.
